(19) United States
(12) Patent Application Publication
(10) Pub. No.: US 2013/0243720 A1
(43) Pub. Date: Sep. 19, 2013

(54) Iron(II)-Containing A Treatments for Hyperphosphatemia

(71) Applicants: Chad Huval, Somerville, MA (US); Pradeep Dhal, Westford, MA (US); Stephen Randall Holmes-Farley, Arlington, MA (US)

(72) Inventors: Chad Huval, Somerville, MA (US); Pradeep Dhal, Westford, MA (US); Stephen Randall Holmes-Farley, Arlington, MA (US)

(21) Appl. No.: 13/534,672

(22) Filed: Jun. 27, 2012

Related U.S. Application Data
(63) Continuation of application No. 12/307,420, filed on Jul. 23, 2009, now abandoned.
(60) Provisional application No. 60/818,727, filed on Jul. 5, 2006.

(51) Int. Cl. A61K 33/26; A61K 31/785; A61K 45/06; A61K 31/295

(52) U.S. Cl. CPC A61K 33/26; A61K 31/295; A61K 31/785; A61K 45/06
USPC 424/78.27; 514/502; 424/646; 424/78.35; 424/648; 424/643

(57) ABSTRACT

A pharmaceutical composition comprises a pharmaceutically acceptable ferrous iron compound; an amine polymer or a pharmaceutically acceptable salt thereof, and a pharmaceutically acceptable carrier. Alternatively, a pharmaceutica composition comprises a pharmaceutically acceptable ferrous iron compound and pharmaceutically acceptable carrier, wherein the ferrous iron compound is selected from the group consisting of iron(II) acetate, iron(II) citrate, iron(II) ascorbate, iron(II) oxalate, iron(II) oxide, iron(II) carbonate, iron(II) carbonate saccharated, iron(II) formate, iron(II) sulfate, iron(II) chloride, iron(II) acetylacetonate and combinations thereof. A method of treating a subject with hyperphosphatemia comprises administering to the subject an effective amount of a pharmaceutical composition as described above.

### RELATED APPLICATION

This application claims the benefit of U.S. Provisional Application No. 60/818,727, filed on Jul. 5, 2006, the entire teachings which are incorporated herein by reference.

### BACKGROUND OF THE INVENTION

People with inadequate renal function, hypoparathyroidism, or certain other medical conditions often have hyperphosphatemia, or elevated serum phosphate levels. Hyperphosphatemia, especially if present over extended periods of time, leads to severe abnormalities in calcium and phosphorus metabolism, often manifested by hyperparathyroidism, bone disease and calcification in joints, lungs, eyes and vasculature. For patients who exhibit renal insufficiency, elevation of serum phosphorus has been associated with progression of renal failure and an increased risk of cardiovascular events.

Oral administration of certain phosphate binders, to bind intestinal phosphate and prevent absorption, has also been suggested. Typical phosphate binders include calcium, aluminum, magnesium and lanthanum compounds. Aluminum-based phosphate binders which have been used for treating hyperphosphatemia includes Amphojel® aluminum hydroxide gel. Other calcium- and aluminum-free phosphate binders have drawbacks including the amount and frequency of dosing required to be therapeutically active.

Polymer materials, such as aliphatic amine polymers, have also been used in the treatment of hyperphosphatemia. These polymers provide an effective treatment for decreasing the serum level of phosphate.

### SUMMARY OF THE INVENTION

In one embodiment, the present invention is directed to a pharmaceutical composition comprising a pharmaceutically acceptable ferrous iron compound and a pharmaceutically acceptable carrier. The ferrous iron compound is selected from the group consisting of iron(II) acetate, iron(II) citrate, iron(II) ascorbate, iron(II) oxalate, iron(II) oxide, iron(II) carbonate, iron(II) carbonate saccharated, iron(II) formate, iron(II) sulfate, iron(II) chloride, iron(II) acetylacetonate and combinations thereof.

In another embodiment, the present invention is directed to a pharmaceutical composition comprising a pharmaceutically acceptable ferrous iron compound, an amine polymer or a pharmaceutically acceptable salt thereof, and a pharmaceutically acceptable carrier.

In yet another embodiment, the present invention is directed to a method of treating a subject with hyperphosphatemia, wherein the method comprises administering to the subject an effective amount of a pharmaceutically acceptable ferrous iron compound. The ferrous iron compound is selected from the group consisting of iron(II) acetate, iron(II) citrate, iron(II) ascorbate, iron(II) oxalate, iron(II) oxide, iron(II) carbonate, iron(II) carbonate saccharated, iron(II) formate, iron(II) sulfate, iron(II) chloride, iron(II) acetylacetonate and combinations thereof.

In yet another embodiment, the present invention is directed to a method of treating a subject with hyperphosphatemia, wherein the method comprises administering to the subject an effective amount of a pharmaceutical composition comprising a pharmaceutically acceptable ferrous iron compound and an aliphatic amine polymer or a pharmaceutically acceptable salt thereof.

### DETAILED DESCRIPTION OF THE INVENTION

As used herein, a “pharmaceutically acceptable ferrous iron compound” means a compound comprising an iron(II) cation, which does not cause unacceptable side effects at the dosages which are being administered. The pharmaceutically acceptable ferrous iron compound can be water-soluble or water-insoluble.

It is to be understood that a “pharmaceutically acceptable ferrous iron compound” encompasses different polymorphs of the pharmaceutically acceptable iron compound. The term “polymorph” refers to solid crystalline forms of a compound. Each polymorph may exhibit different physical, chemical or spectroscopic properties from other polymorphs.

The term “pharmaceutically acceptable ferrous iron compound” also includes various solvates of the pharmaceutically acceptable ferrous iron compound, which include a stoichiometric or non-stoichiometric amount of solvent, e.g., water or organic solvent, bound by non-covalent intermolecular forces.

Preferred pharmaceutically acceptable ferrous iron compounds have a high weight percentage of iron, and/or have a high density, i.e., equal to or greater than 1 g/mL. These iron compounds can minimize daily dose volume.

Examples of pharmaceutically acceptable ferrous iron compounds suitable for the invention include iron(II) acetate, iron(II) citrate, iron(II) ascorbate, iron(II) oxalate, iron(II) oxide, iron(II) carbonate, iron(II) carbonate saccharated, iron(II) formate, iron(II) sulfate, iron(II) chloride, iron(II) acetylacetonate and combinations thereof. When referring to any of these iron compounds, it is to be understood that mixtures, polymorphs and solvates thereof are encompassed.

Examples of preferred pharmaceutically acceptable ferrous iron compounds in the invention include iron(II) oxide, iron(II) acetate, iron(II) citrate, iron(II) ascorbate, iron(II) oxalate and combinations thereof. More preferred pharmaceutically acceptable ferrous iron compounds in the invention include iron(II) oxide and iron(II) acetate.

In some embodiments, the pharmaceutically acceptable ferrous iron compound is a polymer comprising iron(II) (hereinafter “iron(II) binding polymer”). Such iron(II) binding polymers comprise groups that bind or chelate (ionically or covalently) iron(II). Examples of groups which bind or chelate iron(II) include: —COOH; −COO⁻; —OH; —C(O)N(H)OH;

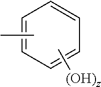

where z is an integer from one to five, such as one, two or three; —C(O)N(H)—(CR′R″)ᵣ—OH where r is zero or an integer from one to ten, such as one, two or three, and R′ and R″ are each independently —H, a substituted or unsubstituted alkyl group or an aryl group, preferably —H; and the like.

In some specific embodiments, the iron(II) binding polymer comprises side chains bonded to the polymer backbone, wherein at least some of the side chains comprise a group(s) which binds or chelates (ionically or covalently) iron(II). Examples of such side chains include —(CR′R″)ᵣ—COOH, —(CR′R″)ᵣ—COO⁻, —(CR′R″)ᵣ—C(O)N(H)OH, —(CR′R″)ᵣ—C(O)N(H)—(CR′R″)ᵣ—OH,

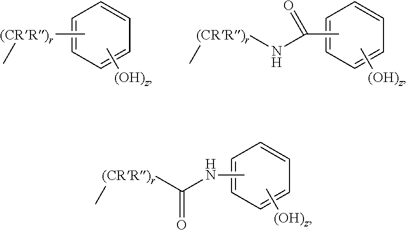

where each of R′ and R″ is independently —H, a substituted or unsubstituted alkyl group or an aryl group, preferably —H; each r is independently an integer from one to ten, such as one, two or three; and each z is independently an integer from one to five. Specific examples of iron(II) binding polymers include, but are not limited to, those described in the following paragraphs.

One example of an iron(II) binding polymer is a poly(acrylic acid) or a salt thereof, such as sodium, potassium or ammonium salt, or a mixed salt thereof.

Another example of an iron(II) binding polymer is a hydroxylamine- or hydroxyalkylamine-modified poly(alkylacrylate-co-divinylbenzene), such as poly(2-hydroxyethylacrylate-co-divinylbenzene), wherein some of the carboxylate groups of the poly(alkylacrylate-co-divinylbenzene) are modified with hydroxylamine or hydroxyalkylamine to form N-hydroxylamide groups or N-hydroxyalkylamide groups.

Another example of an iron(II) binding polymer is a hydroxylamine- or hydroxyalkylamine-modified poly(alkylacrylate), wherein some of the acrylate groups are amidated with an amine group of an amine polymer. A specific example of an iron(II) binding polymer of this type comprises a repeat unit represented by Structural Formula (1):

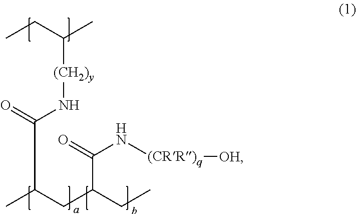

or a salt thereof, where y and q are each independently zero or an integer from one to ten, such as one, two or three; a and b are each independently a positive integer; R′ and R″ are each independently —H, a substituted or unsubstituted alkyl group or an aryl group, preferably —H. Preferably, y and q are zero or one, more preferably y is one and q is zero, and a and b are selected to have a molecular weight as described below for amine polymers. The amine polymer is preferably a polyallylamine polymer, more preferably a polyallylamine homopolymer.

Another example of an iron(II) binding polymer is an amine polymer, preferably an aliphatic amine polymer, modified with an alkylacrylate (e.g., ethylacrylate). A specific example of an iron(II) binding polymer of this type comprises a repeat unit represented by Structural Formula (2), (3) or (4):

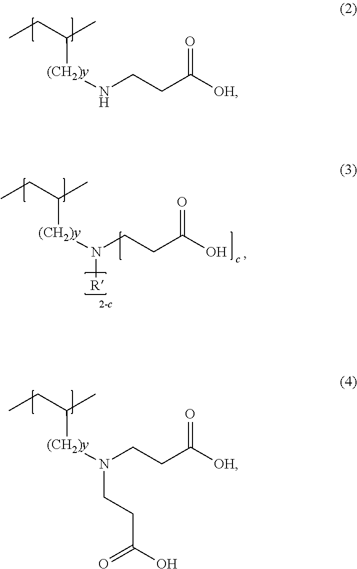

or a salt thereof, where y is zero or an integer from one to ten, such as one, two or three; R′ is —H, a substituted or unsubstituted alkyl group or an aryl group, preferably —H; and c is one or two. Preferably, y is one. The amine polymer is preferably a polyallylamine polymer, more preferably a polyallylamine homopolymer.

Another example of an iron(II) binding polymer is an amine polymer, preferably an aliphatic amine polymer, modified with hydroxylamine or a hydroxyalkylamine. A specific example of an iron(II) binding polymer of this type comprises a repeat unit represented by Structural Formula (5), (6), (7) or (8):

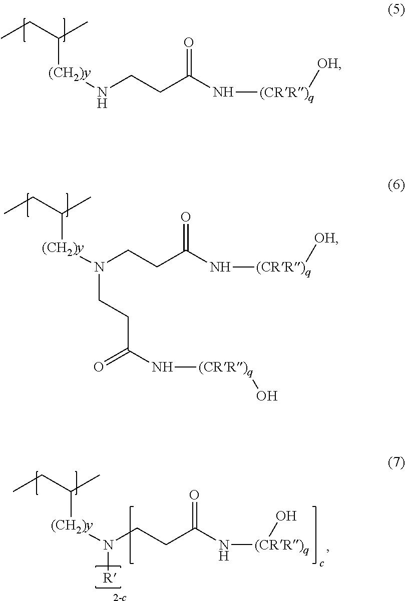

or a salt thereof, where y and q are each independently zero or an integer from one to ten, such as one, two or three; R′ and R″ are each independently —H, a substituted or unsubstituted alkyl group or an aryl group, preferably —H; and c is one or two. Preferably, y and q are zero or one, more preferably y is one and q is zero. The amine polymer is preferably a polyallylamine polymer, more preferably a polyallylamine homopolymer.

Another example of an iron(II) binding polymer is an amine polymer, preferably an aliphatic amine polymer, modified with a mono-, di-, tri-, tetra- or penta-hydroxybenzoic acid (e.g., 3,4-dihydroxybenzoic acid), wherein some amine groups of the amine polymers have been benzoylated with the mono-, di-, tri-, tetra- or penta-hydroxybenzoic acid. Yet another example of an iron(II) binding polymer is an amine polymer, preferably an aliphatic amine polymer, wherein at least some amine groups of the amine polymer are benzylated with the mono-, di-, tri-, tetra- or penta-hydroxybenzyl group (e.g., a 3,4-dihydroxybenzyl group). Specific examples of iron(II) binding polymers of these types comprise a repeat unit represented by Structural Formula (8) or (9):

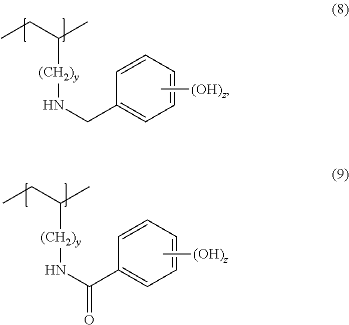

where y is zero or an integer of one to ten, preferably between one and three, more preferably one; and z is an integer of one to five, such as one, two or three.

The iron(II) binding polymers can be optionally crosslinked with a crosslinking agent. Examples of suitable crosslinking agents and of degree of crosslinking are as described below for amine polymers.

The iron(II) binding polymers can be used in the invention alone or in combination with the pharmaceutically acceptable ferrous iron compounds described above. When the iron(II) binding polymers are used in combination with the pharmaceutically acceptable ferrous iron compounds described above, the ferrous iron compounds may optionally be entrained within the polymers. As used herein, the phrase “ferrous iron compound entrained within the iron(II) binding polymer” means that the ferrous iron compound or the ferrous ion of the ferrous iron compound is encaptured within the polymer, for example, within a polymeric network, such as a pocket (or pockets) of the polymer created by crosslinking.

Optionally, the pharmaceutical composition of the invention comprises a mixture of pharmaceutically acceptable ferrous iron compounds. Suitable examples of the pharmaceutically acceptable ferrous iron compounds for the composition are as described above.

Preferably, the pharmaceutical compositions of the invention are essentially free of ferric ion. As used herein, the term “pharmaceutical composition essentially free of ferric ion” means that the pharmaceutical composition has a ferric ion content that is less than 10% by mole, preferably less than 5% by mole or more preferably less than 1% by mole of the total iron content of the pharmaceutical composition.

Preferably, the pharmaceutically acceptable ferrous iron compound is administered in the substantial absence of ferric ion. As used herein, the term “administered in the substantial absence of ferric iron” means that when the ferrous iron compound is administered to the subject, less than 10% by mole, preferably less than 5% by mole or more preferably less than 1% by mole, of the total iron content being administered to the subject is ferric iron.

The present invention also includes a pharmaceutical composition comprising a pharmaceutically acceptable carrier; a pharmaceutically acceptable ferrous iron compound; and a phosphate sequestrant.

As used herein, the term “phosphate sequestrant” means a pharmaceutically acceptable compound other than a pharmaceutically acceptable ferrous iron compound which binds phosphate. The phosphate sequestrant can be a calcium-, aluminum-, magnesium-, zinc- or lanthanum-containing phosphate binder or a phosphate-binding amine polymer such as those disclosed in U.S. Pat. Nos. 5,496,545; 5,667,775 and 6,083,495 (the contents of which are incorporated herein by reference in their entirety). Preferably, the phosphate-binding amine polymer is an aliphatic amine polymer.

Amine polymers are characterized by a repeat unit that includes at least one amine group. Amine groups can be part of the polymer backbone (e.g., a polyalkyleneimine such as polyethyleneimine), pendant from the polymer backbone (e.g., polyallylamine), or both types of amine groups can exist within the same repeat unit and/or polymer. Amine polymers include aliphatic amine polymers and aromatic amine polymers. The word “amine,” as used herein, includes primary, secondary and tertiary amines, as well as ammonium groups such as trialkylammonium.

Aromatic amine polymers are characterized by a repeat unit comprising an amine attached to the polymer backbone by an aromatic group (e.g., phenylene) or a linking group comprising an aromatic group (e.g., alkylene-phenylene, phenylene-alkylene or alkylene-phenylene-alkylene. Examples of aromatic amine polymers include poly(vinylbenzyl trimethylammonium chloride) and polystyrene trimethylbenzylammonium chloride. A specific example of aromatic amine polymer is cholestyramine.

Aliphatic amine polymers are characterized by a repeat unit comprising an amine group attached to the polymer backbone of the polymer by an aliphatic group, or an amine group that is a part of the polymer backbone where the polymer backbone is non-aromatic. An aliphatic amine polymer may be obtained by polymerizing an aliphatic amine monomer. An aliphatic amine monomer is an amine group attached to a polymerizable group such as an olefin by an aliphatic group. Suitable aliphatic amine polymers are described in U.S. Pat. Nos. 5,487,888, 5,496,545, 5,607,669, 5,618,530, 5,624,963, 5,667,775, 5,679,717, 5,703,188, 5,702,696, 5,693,675, 5,900,475, 5,925,379, 6,083,497; 6,177,478, 6,083,495, 6,203,785, 6,423,754, 6,509,013, 6,605,270, 6,726,905, 6,733,780 and 6,858,203 and U.S. Published Applications Nos. 2002/0159968 A1 and 2003/0086898 A1, the contents of which are incorporated herein by reference in their entireties.

An aliphatic amine polymer may be a homopolymer or a copolymer of one or more aliphatic amine monomers or a copolymer of one or more aliphatic amine monomers in combination with one or more monomers which do not comprise an amine and are preferably inert and non-toxic. Examples of suitable monomers which do not comprise an amine include vinyl alcohol, acrylic acid, acrylamide, and vinylformamide. Alternatively, an aliphatic amine polymer can be a co-polymer of two or more different aliphatic amine monomers.

Examples of aliphatic amine polymers include polymers that have one or more repeat units selected from Formulas (10)-(15):

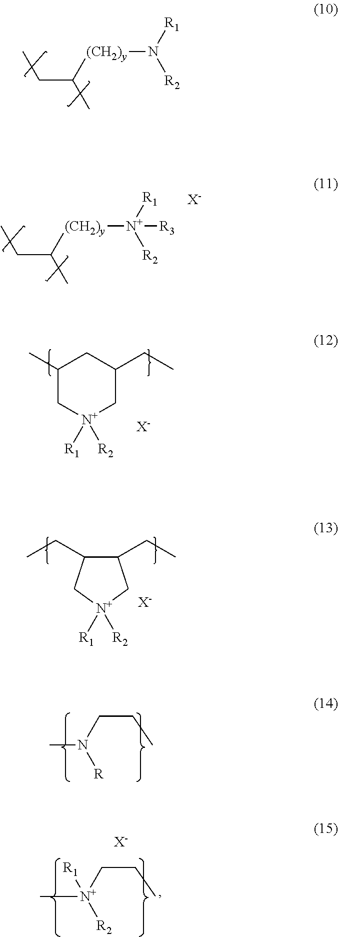

or a salt or copolymer thereof, where y is zero or an integer of one or more (e.g., between about one and about 10, preferably between one and four, more preferably one) and each R, R₁, R₂, and R₃, independently, is 1-1, a substituted or unsubstituted alkyl group (e.g., having between 1 and 25 or between 1 and 5 carbon atoms, inclusive) or aryl (e.g., phenyl) group, and each X is an exchangeable negatively charged counterion.

Preferably, at least one of R, R₁, R₂, or R₃ is a hydrogen atom. More preferably, each of these groups is hydrogen.

The alkyl or aryl group, represented by R, R₂, and R₃, can carry one or more substituents. Suitable substituents include cationic groups, e.g., quaternary ammonium groups, or amine groups, e.g., primary, secondary or tertiary alkyl or aryl amines. Examples of other suitable substituents include hydroxy, alkoxy, carboxamide, sulfonamide, halogen, alkyl, aryl, hydrazine, guanidine, urea, poly(alkyleneimine) such as poly(ethylenimine), and carboxylic acid esters.

Preferably, an aliphatic amine polymer for use in the invention is a homopolymer, such as a homopolyallylamine polymer, a homopolyvinylamine polymer, a homopolydiallylamine polymer or a polyethyleneamine polymer. Alternatively, an aliphatic amine polymer for use in the invention is can also be a co-polymer.

In one embodiment, the aliphatic amine polymer is a homopolymer or copolymer characterized by one or more repeat units of Structural Formula (16):

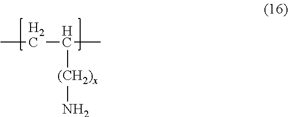

or a pharmaceutically acceptable salt thereof, where x is 0 or an integer between 1 and 4, preferably 1. The polymer represented by Structural Formula (16) is advantageously crosslinked by means of a crosslinking agent.

A preferred aliphatic amine polymer for use in the invention is a polyallylamine polymer, which is a polymer having repeat units from polymerized allylamine monomers. The amine group of an allylamine monomer can be unsubstituted or substituted with, for example, one or two C1-C10 straight chain or branched alkyl groups. These alkyl groups are optionally substituted with one or more hydroxyl, amine, halo, phenyl, amide or nitrile groups. Preferably, the aliphatic amine polymers that may be used in the present invention are polyallylamine polymers comprising repeat units represented by Structural Formula (17):

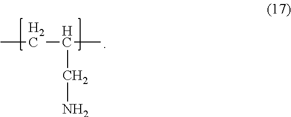

Polyallylamine polymers that may be used in the present invention may include copolymers comprising repeat units from two or more different polymerized allylamine monomers or with repeat units from one or more polymerized allylamine monomers and repeat units from one or more polymerized monomers which are not allylamines. Examples of suitable monomers which are not allylamines include acrylamide monomers, acrylate monomers, maleic acid, maleimide monomers, vinyl acylate monomers and alkyl substituted olefines. Alternatively, other olefinic aliphatic amine monomers can be polymerized with an allylamine monomer. Preferably, however, the polyallylamine polymers used in the present invention comprise repeat units solely from polymerized allylamine monomers. More preferably, the polyallylamine polymers used in the present invention are homopolymers. Even more preferably, the polyallylamine polymers used in the present invention are homopolymers of repeat units represented by Structural Formula (17). Polyallylamine polymers used in the disclosed invention are preferably crosslinked polymers, more preferably crosslinked homopolymers.

Another preferred aliphatic amine polymer for use in the invention is a polyvinylamine polymer, which is a polymer having repeat units from polymerized vinylamine monomers. The amine group of an vinylamine monomer can be unsubstituted or substituted with, for example, one or two C1-C10 straight chain or branched alkyl groups. These alkyl groups are optionally substituted with one or more hydroxyl, amine, halo, phenyl, amide or nitrile groups. Examples of vinylamine monomers include N-vinylformamide, N-vinylurea, 1-vinylimidazole, 1-vinyl-1,2,4-triazole, N-methyl-N-vinylacetamide, Trimethylvinylammonium hydroxide, 1-vinyl-2-pyrrolidinone, N-vinylsuccinimide, N-vinyl-2-piperidone, 2-hydroxyethylethylene urea, N,N-di vinylethyleneurea, N-vinylcaprolactam, (N-vinylformamido)trimethylsilane, trimethylvinylammonium bromide, N-vinylphthalimide and Benzyl-N-vinylcarbamate. Polyvinylamine polymers that may be used in the present invention may include copolymers comprising repeat units from two or more different polymerized vinylamine monomers or with repeat units from one or more polymerized vinylamine monomers and repeat units from one or more polymerized monomers which are not vinylamines.

Other examples of aliphatic amine polymers suitable for use in the invention are copolymers of diethylenetriamine, preferably crosslinked by means of a multifunctional crosslinking agent. Preferably, the aliphatic amine polymer is an epichlorohydrin-crosslinked copolymer of di ethylenetriamine, such as colestipol.

In other embodiments, the amine polymers suitable for use in the invention can be a homopolymer or copolymer of polybutenylamine, polylysine, or polyarginine.

Amine polymers (aliphatic and aromatic amine polymers) are typically crosslinked with crosslinking agents. Preferably, the amine polymers are rendered water-insoluble by crosslinking such as with a crosslinking agent. Suitable crosslinking agents include those with functional groups which react with the amine group of the amine monomer. Alternatively, the crosslinking agent may contain-two or more vinyl groups which undergo free radical polymerization with the amine monomer. In some cases the amine polymers are crosslinked after polymerization.

Examples of suitable crosslinking agents include diacrylates and dimethylacrylates (e.g., ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate and polyethyleneglycol diacrylate), methylene bisacrylamide, methylene bismethacrylamide, ethylene bisacrylamide, ethylene bismethacrylamide, ethylidene bisacrylamide, divinylbenzene, bisphenol A, the diglycidyl ether of bisphenol A, pyromellitic dianhydride, toluene diisocyanate, ethylene diamine and dimethyl succinate, dimethacrylate, and bisphenol A diacrylate. Examples of preferred difunctional crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride. Epichlorohydrin is a most preferred crosslinking agent, because of its high availability and low cost. Epichlorohydrin is also advantageous because of its low molecular weight and hydrophilic nature, increasing the water-swellability and gel properties of the polyamine. Epichlorohydrin forms 2-hydroxypropyl crosslinking groups.

Other methods of inducing crosslinking on already polymerized materials include, but are not limited to, exposure to ionizing radiation, ultraviolet radiation, electron beams, radicals, and pyrolysis.

The level of crosslinking renders the crosslinked amine polymers insoluble and substantially resistant to absorption and degradation, thereby limiting the activity of the crosslinked amine polymers to the gastrointestinal tract, and reducing potential side-effects in the patient. Typically, the crosslinking agent is present in an amount 0.5-35% (such as 0.5-25%, 2.5-20% or 1-10%) by weight, based upon total weight of amine monomer plus crosslinking agent.

Typically, between 1% by mole and 30% by mole of the allylic nitrogen atoms are bonded to a crosslinking group, preferably between 6% by mole and 21% by mole.

The amine polymers can also be further derivatized; examples include alkylated amine polymers, as described, for example, in U.S. Pat. Nos. 5,679,717, 5,607,669 and 5,618,530, the teachings of which are incorporated herein by reference in their entireties. Preferred alkylating agents include hydrophobic groups (such as aliphatic hydrophobic groups) and/or quaternary ammonium- or amine-substituted alkyl groups. Examples of suitable alkylating agents include a C₁-C₂₀ alkyl halide (e.g., an n-butyl halide, n-hexyl halide, n-octyl halide, n-decyl halide, n-dodecyl halide, n-tetradecyl halide, n-octadecyl halide, and combinations thereof); a C₁-C₂₀ dihaloalkane (e.g., a 1,10-dihalodecane); a C₁-C₂₀ hydroxyalkyl halide (e.g., an 11-halo-1-undecanol); a C₁-C₂₀ aralkyl halide (e.g., a benzyl halide); a C₁-C₂₀ alkyl halide ammonium salt (e.g., a (4-halobutyl) trimethylammonium salt, (6-halohexyl)trimethyl-ammonium salt, (8-halooctyl)trimethylammonium salt, (10-halodecyl)trimethylammonium salt, (12-halododecyl)-trimethylammonium salts and combinations thereof); a C₁-C₂₀ alkyl epoxy ammonium salt (e.g., a (glycidylpropyl)-trimethylammonium salt); a C₁-C₂₀ epoxy alkylamide (e.g., an N-(2,3-eoxypropane)butyramide, N-(2,3-epoxypropane) hexanamide, and combinations thereof); and a haloalkylamine compounds (e.g., 2-bromoethylamine hydrobromide, 2-chloroethylamine hydrochloride, 3-bromopropylamine hydrobromide, 3-chloropropylamine hydrochloride, 3-chloropropylamine, 4-bromobutylamine hydrobromide, 4-chlorobutylamine hydrochloride, 5-bromo-1-pentylamine hydrochloride, 5-chloropentylamine and 6-bromohexylamine hydrochloride).

Non-crosslinked and crosslinked polyallylamine polymers and polyvinylamine polymers are generally known in the art and are commercially available. Methods for the manufacture of polyallylamine polymers and polyvinylamine polymers, and crosslinked derivatives thereof, are described in the above U.S. Patents. Patents by Harada et al., (U.S. Pat. Nos. 4,605,701 and 4,528,347), which are incorporated herein by reference in their entireties, also describe methods of manufacturing polyallylamine polymers and crosslinked polyallylamine polymers. A patent by Stuffs et al., (U.S. Pat. No. 6,180,754) describes an additional method of manufacturing crosslinked polyallylamine polymers.

The molecular weight of amine polymers is not believed to be critical, provided that the molecular weight is large enough so that the amine polymers are substantially non-absorbed by the gastrointestinal tract. Typically, the molecular weight of amine polymers, preferably aliphatic amine polymers, is at least 1000. For example, the molecular weight can be from: about 1000 to about 5 million, about 1000 to about 3 million, about 1000 to about 2 million, or about 1000 to about 1 million.

The amine polymers used in the invention may be fully protonated or fully unprotonated. Alternatively, the amine polymers used in the invention may be partially protonated, and in one embodiment, include amine polymers in which less than 50% by mole, for example, less than 40%, less than 30%, less than 20% or less than 10%, of the amine groups are protonated. In another embodiment, 35% to 45% by mole of the amines are protonated (e.g., approximately 40% by mole). An example of a suitably protonated amine polymer is sevelamer hydrochloride.

As described above, the amine polymer can be administered in the form of a pharmaceutically acceptable salt. The term “pharmaceutically acceptable salt” of an amine polymer refers to a salt of the amine polymer to be administered which is prepared from pharmaceutically acceptable non-toxic acids including inorganic acids, organic acids, solvates, hydrates, or clathrates thereof. Thus, the nitrogen group in the repeat unit of the amine polymer is protonated to create a positively charged nitrogen atom associated with a negatively charged counterion. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

Examples of suitable counterions (e.g., suitable counterions for X⁻ in Structural Formulas (11)-(13) and (15)) include organic ions, inorganic ions, or a combination thereof. For instance, suitable counterions include halides (e.g., F⁻, Cl⁻, Br⁻ and I⁻), CH₃OSO₃⁻, HSO₄⁻, SO₄²⁻, HCO₃⁻, CO₃²⁻, acetate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Preferred anions are Cl⁻, HCO₃⁻, CO₃²⁻, or a combination thereof (e.g., a mixed carbonate and bicarbonate salt, a mixed carbonate and chloride salt, or a mixed bicarbonate and chloride salt). The counterions can be the same as, or different from, each other. For example, the amine polymer can contain two or more different types of counterions.

In a preferred embodiment, the amine polymer used in the present invention is an epichlorohydrin-crosslinked polyallylamine polymer, such as sevelamer and colesevelam (see, for example, U.S. Pat. Nos. 6,423,754; 5,607,669; and 5,679,717, the contents of which are incorporated herein by reference). In a more preferred embodiment, the polyallylamine polymer is crosslinked with epichlorohydrin and between about 9% to about 30% by weight (preferably about 15% to about 21% by weight) of the allylic nitrogen atoms are bonded to a crosslinking group and the anion is chloride, carbonate or bicarbonate or a mixed salt thereof.

A particularly preferred amine polymer is polyallylamine hydrochloride crosslinked with about 9.0-9.8% w/w epichlorohydrin, preferably 9.3-9.5%, and is the active chemical component of the drug known as sevelamer HCl, sold under the tradename RENAGEL®. The structure is represented below:

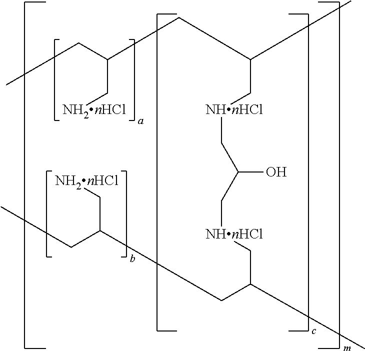

where:

the sum of a and b (the number of primary amine groups) is 9;

c (the number of crosslinking groups) is 1;

n (the fraction of protonated amines) is 0.4; and

m is a large number (to indicate extended polymer network).

Another particularly preferred amine polymer is a polyallylamine hydrochloride crosslinked with epichlorohydrin, and alkylated with 1-bromodecane and (6-bromohexyl)-trimethylammonium bromide, referred to as colesevelam HCl, and marketed in the United States as WELCHOL®.

In yet another particularly preferred embodiment, the amine polymer is a carbonate salt of sevelamer; a bicarbonate salt of sevelamer; a mixed carbonate and bicarbonate salt of sevelamer; or a mixed carbonate and chloride salt of sevelamer.

In other embodiments, a monovalent anionic source is mixed with a carbonate salt of an aliphatic amine polymer. Various examples of carbonate salts of the aliphatic amine polymer and monovalent anionic sources are disclosed in U.S. application Ser. No. 11/262,291, filed Oct. 27, 2005 and U.S. application Ser. No. 11/262,291, filed Oct. 27, 2005, the entire contents of which are incorporated herein by reference.

The monovalent anion comprises at least 0.01%, preferably 0.05%, more preferably a range of 0.01% to 2%, 0.05% to 1%, 0.08% to 0.5%, or 0.1% to 0.3% by weight of the combined weights of the carbonate salt of an aliphatic amine polymer and the monovalent anion source.

Examples of suitable monovalent anions include organic ions, inorganic ions, or a combination thereof, such as halides (Cl⁻, I⁻, F⁻ and Br⁻), CH₃OSO₃⁻, HSO₄⁻, acetate, lactate, butyrate, propionate, sulphate, citrate, tartrate, nitrate, sulfonate, oxalate, succinate or palmoate. Preferred monovalent anions are halides, most preferably chloride.

Also, the monovalent anion source can be a pharmaceutically acceptable acid, ammonium or metal salt of a monovalent anion. Preferred examples of the monovalent anion source include sodium chloride and hydrochloric acid. In one preferred embodiment, the formulations of the invention comprise a carbonate salt of sevelamer and sodium chloride. In another preferred embodiment, the formulations of the invention comprise a carbonate salt of sevelamer and hydrochloric acid.

In yet another embodiment, when a carbonate salt of an aliphatic amine polymer is included in the invention, the monovalent anion source can be a monovalent anion salt of an aliphatic amine polymer comprising a repeat unit represented by Structural Formulas (10)-(17) above. In this embodiment, a monovalent anion salt of an aliphatic amine polymer and the carbonate salt of an aliphatic amine polymer can be physically mixed together. Alternatively, a single aliphatic amine polymer can comprise both carbonate and monovalent anions to form a mixed carbonate and monovalent anion salt of the single aliphatic amine polymer. When a monovalent anion salt of an aliphatic amine polymer and a carbonate salt of an aliphatic amine polymer are physically mixed together, the monovalent anion salt of an aliphatic amine polymer can be the same or a different aliphatic amine polymer as the aliphatic amine polymer carbonate salt.

An “aliphatic group” is non-aromatic, consists solely of carbon and hydrogen and may optionally contain one or more units of unsaturation, e.g., double and/or triple bonds. An aliphatic group may be straight chained, branched or cyclic. Unless otherwise provided, a straight chained or branched aliphatic group contains between 1 and 10 carbon atoms, preferably between 1 and 3 carbon atoms. Unless otherwise provided, a cyclic aliphatic group contains between 3 and 8 carbon atoms. Suitable substituents for an aliphatic group include amine groups, e.g., primary, secondary or tertiary alkyl amines. Examples of other suitable substituents include hydroxy, alkoxy, carboxamide, sulfonamide, halogen, alkyl, aryl, hydrazine, guanidine and urea.

The term “alkyl”, used alone or part of larger moiety such as “alkoxy”, “alkylamine”, “hydroxyalkylamine”, “dialkyamine”, means a saturated aliphatic group. Suitable substituents for an alkyl group are as defined above for an aliphatic group.

“Alkylene” is a bivalent alkyl group, i.e., —(CH₂)ₙ—, wherein n is an integer from 1-10, preferably 1-3.

“Aromatic groups” include monocyclic carbocyclic and heterocyclic aromatic groups such as phenyl, pyridyl, thienyl, furanyl, and the like. “Phenylene” is a bivalent phenyl group, i.e.,

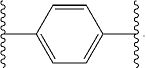 [SMILES: *c1ccc(*)cc1]

Suitable substituents for an aromatic group are as described for an aliphatic group.

When an amine polymer as described above is used in combination with a pharmaceutically acceptable ferrous iron compound of the invention, the amine polymer and the pharmaceutically acceptable ferrous iron compound can be co-formulated in a single pharmaceutical composition, or alternatively co-administered in separate pharmaceutical compositions.

In some embodiments, the amine polymer, preferably an aliphatic amine polymer, and the pharmaceutically acceptable ferrous iron compound are co-formulated in a single pharmaceutical composition. The amine polymer and the pharmaceutically acceptable ferrous iron compound can be present in an admixture thereof. Alternatively, the pharmaceutically acceptable ferrous iron compound can be entrained within a crosslinked amine polymer, preferably a crosslinked aliphatic amine polymer, as described above. As used herein, the phrase “pharmaceutically acceptable ferrous iron compound entrained within a crosslinked amine polymer” means that the crosslinked amine polymer encaptures the pharmaceutically acceptable ferrous iron compound or the ferrous ion of the iron compound, for example, within a pocket (or pockets) generated by crosslinking. A pharmaceutically acceptable ferrous iron compound entrained with a crosslinked amine polymer, preferably a crosslinked aliphatic amine polymer, can be prepared by crosslinking an amine polymer as described above in the presence of a pharmaceutically acceptable ferrous iron compound. For example, a polyallylamine polymer can be crosslinked by multifunctional crosslinking agent(s), such as epichlorohydrin, in the presence of iron(II) oxide to form a crosslinked polyallylamine polymer entraining iron(II) oxide or the iron(II) cation of the iron(II) oxide. Various examples and preferred values for the amine polymers, crosslinking agents and pharmaceutically acceptable ferrous iron compounds are as described above. Typically, when a pharmaceutically acceptable ferrous iron compound entrained with a crosslinked amine polymer, preferably a crosslinked aliphatic amine polymer, is employed, the crosslinking agent is present in an amount 0.5-35% (such as 0.5-30%, 2.5-30%, 5-25%, 5-20% or 5-15%) by weight, based upon total weight of amine monomer plus crosslinking agent.

When the amine polymer, preferably an aliphatic amine polymer, and a pharmaceutically acceptable ferrous iron compound are formulated in a single pharmaceutical composition, typically, the ferrous ion of the pharmaceutically acceptable ferrous iron compound comprises 5-35%, such as 10-30%, 10-25%, 13-25%, 15-22% and 16-20%, by anhydrous weight of the pharmaceutical composition.

Alternatively, the ferrous ion of the pharmaceutically acceptable ferrous iron compound comprises 5-35%, such as 1.0-30%, 10-25%, 13-25%, 15-22% and 16-20%, by anhydrous weight of the combined weight of the ferrous iron compound and the free base of the amine polymer. Herein, the term “the free base of the amine polymer” means the amine polymer not including any counter ion. When the quantity of ferrous iron compound in the pharmaceutical composition is expressed in this fashion, it is to be understood that the amine polymer in the pharmaceutical composition can be unprotonated, partially protonated or completely protonated. However, the weight of the amine polymer is calculated assuming it is the corresponding free base amine polymer and that all of the nitrogen atoms in the amine polymer are free and not bound to any counter ions.

Alternatively, the pharmaceutically acceptable ferrous iron compound is present in the pharmaceutical composition in an amount such that the molar ratio of the ferrous ion of the pharmaceutically acceptable ferrous iron compound to the total amine nitrogen atoms (protonated and unprotonated) of the polymer is 0.1-3.0, such as 0.4-3.0, 0.4-2.5, 0.8-2.0, 0.8-1.5 and 0.8-1.3. Preferably, the molar ratio is 1. This ratio is the quotient of moles of ferrous ion of the pharmaceutically acceptable ferrous iron compound to moles of nitrogen atom in the amine polymer. If present, nitrogen from a counter ion or cross-linker is included in the moles of the amine polymer.

Alternatively, the pharmaceutically acceptable ferrous iron compound is present in the pharmaceutical composition in an amount such that the weight ratio of the ferrous ion of the pharmaceutically acceptable ferrous iron compound to the total nitrogen atoms of the amine polymer is 0.7-2.5, such as 0.7-2.0, 1.0-2.0 and 1.2-1.8. Preferably, the weight ratio is 1.57. This weight ratio is the quotient of grams of ferrous ion to grams of nitrogen atoms in the amine polymer (but not the entire composition). Thus, nitrogen from a counter ion or cross-linker, if present, is included in the grams of the nitrogen atoms in the amine polymer.

Alternatively, the pharmaceutically acceptable ferrous iron compound is present in the pharmaceutical composition in an amount such that the weight ratio of the ferrous ion of the pharmaceutically acceptable ferrous iron compound to the free base of the aliphatic amine polymer is 0.2-1.2, such as 0.2-1.0, 0.3-1.0, 0.3-0.8 and 0.3-0.5. Preferably, the weight ratio is 0.42. The term “the free base of the amine polymer” is as described above. Thus, this ratio is the quotient of grams of ferrous ion to grams of amine polymer not including any weight from any counter ion in the amine polymer.

Other phosphate sequestrants suitable for use in the present invention include pharmaceutically acceptable lanthanum, calcium, aluminum, magnesium and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids, have been utilized for phosphate binding. The ingested calcium combines with phosphate to form insoluble calcium phosphate salts such as Ca₃(PO₄)₂, CaHPO₄, or Ca(H₂PO₄)₂.

Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient.

The most commonly used lanthanide compound, lanthanum carbonate (Fosrenol®) behaves similarly to calcium carbonate.

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Various examples of pharmaceutically acceptable zinc compounds are described in PCT Application No. PCT/US2005/047582 filed Dec. 29, 2005, the entire teachings of which are incorporated herein by references. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbarnate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants (e.g., a mixture of a pharmaceutically acceptable magnesium compound and an amine polymer, preferably an aliphatic amine polymer) described above can be used in the invention in combination with the pharmaceutically acceptable ferrous iron salts.

In other embodiments, the phosphate sequestrant used in combination with the pharmaceutically acceptable ferrous ion compound described above is not a pharmaceutically acceptable magnesium compound. In yet other embodiments, the phosphate sequestrant used in combination with the pharmaceutically acceptable ferrous ion compound described above is not a pharmaceutically acceptable zinc compound.

The invention also includes methods and pharmaceutical compositions directed to a combination therapy of the pharmaceutically acceptable ferrous iron compound described above in combination with a phosphate transport inhibitor; an HMG-CoA reductase inhibitor, such as a statin; or an alkaline phosphatase inhibitor. The invention also includes methods and pharmaceutical compositions directed to a combination therapy of the pharmaceutically acceptable ferrous iron compound described above in combination with a bile acid sequestrant. A mixture of the pharmaceutically acceptable ferrous iron compounds can be employed in the combination therapy with a phosphate transport inhibitor; an HMG-CoA reductase inhibitor; an alkaline phosphatase inhibitor, or a bile acid sequestrant. Suitable pharmaceutically acceptable ferrous iron compounds for the therapy are as described above.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

Suitable examples of HMG-CoA reductase inhibitors for the combination therapy of the invention include lovastatin (mevinolin) (e.g., Altocor® and Mevacor®) and related compounds; pravastatin (e.g., Pravachol®, Selektine®, and Lipostat®) and related compounds; simvastatin (e.g., Zocor®) and related compounds. Other HMG-CoA reductase inhibitors which can be employed in the present invention include fluvastatin (e.g., Lescol®); cerivastatin (e.g., Baycol® and Lipobay®); atorvastatin (e.g., Zarator® and Lipitor®); pitavastatin; rosuvastatin (visastatin) (e.g., Crestor®); guinoline analogs of mevalonolactone and derivatives thereof (see U.S. Pat. No. 5,753,675, the entire teachings of which are incorporated herein by reference); pyrazole analogs of mevalonolactone derivatives (see U.S. Pat. No. 4,613,610, the entire teachings of which are incorporated herein by reference); indene analogs of mevalonolactone derivatives (see WO 86/03488, the entire teachings of which are incorporated herein by reference); 6-[2-(substituted-pyrrol-1-yl)-alkyl)pyran-2-ones and derivatives thereof (see U.S. Pat. No. 4,647,576, the entire teachings of which are incorporated herein by reference); imidazole analogs of mevalonolactone (see WO 86/07054, the entire teachings of which are incorporated herein by reference); 3-hydroxy-4(dihydroxooxophosphorio)butanoic acid derivatives (see French Patent No. 2,596,393, the entire teachings of which are incorporated herein by reference); naphthyl analogs of mevalonolactone (see U.S. Pat. No. 4,686,237, the entire teachings of which are incorporated herein by reference); octahydronaphthalenes (see U.S. Pat. No. 4,499,289, the entire teachings of which are incorporated herein by reference); and quinoline and pyridine derivatives (see U.S. Pat. Nos. 5,506,219 and 5,691,322, the entire teachings of which are incorporated herein by reference). A statin, such as atorvastatin, fluvastatin, lovastatin, pravastatin, simvastatin, rosuvastatin, cerivastatin and pitavastatin, is preferred.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl) imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

Suitable examples of bile acid sequestrants include colesevelam, cholestyramine, and colestipol. Other examples of bile acid sequestrants are disclosed in U.S. Pat. Nos. 5,929,184; 6,129,910; 6,190,649; 6,203,785; 6,271,264; and 6,294,163, the entire teachings of which are incorporated herein by reference.

The pharmaceutical compositions of the invention can be formulated as a tablet, sachet, slurry, food formulation, troche, capsule, elixir, suspension, syrup, wafer, chewing gum or lozenge.

A syrup formulation generally consists of a suspension or solution of the phosphate binding polymer or salt in a liquid carrier, for example, ethanol, glycerine or water, with a flavoring or coloring agent.

Where the pharmaceutical compositions are in the form of a tablet, one or more pharmaceutical carriers routinely used for preparing solid formulations can be employed. Examples of such carriers include magnesium stearate, starch, lactose and sucrose. For example, tablet formulations for oral use can be obtained by combining the active compound with a one or more excipients, optionally grinding a resulting mixture, and processing the mixture of granules, after adding suitable auxiliaries, if desired, to obtain tablet cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Where the pharmaceutical compositions are in the form of a capsule, the use of routine encapsulation is generally suitable, for example, using the aforementioned carriers in a hard gelatin capsule shell. For example, methods for encapsulating compositions (such as in a coating of hard gelatin or cyclodextran) are known in the art (Baker, et al., “Controlled Release of Biological Active Agents”, John Wiley and Sons, 1986, the entire teachings of which are incorporated herein by reference). Where the pharmaceutical compositions are in the form of a soft gelatin shell capsule, carriers routinely used for preparing dispersions or suspensions can be considered, for example, aqueous gums, celluloses, silicates or oils, and are incorporated in a soft gelatin capsule shell. The pharmaceutical compositions can also be in the form of a push-fit capsule made of a suitable material, such as gelatin, as well as soft, sealed capsule made of a suitable material, for example, gelatin, and a plasticizer, such as glycerol or sorbitol. The push-fit capsules can contain the pharmaceutically acceptable ferrous iron compound in admixture with filler such as lactose, binders such as starches, and/or lubricants, such as talc, and, optionally, stabilizers. In soft capsules, the pharmaceutically acceptable ferrous iron compound can be dissolved or suspended in suitable liquids, such as fatty oils, liquid paraffin, or liquid polyethylene glycols. In addition, stabilizers can optionally be added.

In a preferred embodiment, the pharmaceutical compositions of the invention are formulated as a tablet.

In another preferred embodiment, the pharmaceutical compositions of the invention are formulated as a powder formulation which can be easily packaged as a sachet or a tub from which a unit dose is measured by, e.g., a spoon or cup, or an instrument capable of dispensing a pre-defined dosage amount. The powder formulation preferably further includes a pharmaceutically acceptable anionic polymer, such as alginate (e.g., sodium alginate, potassium alginate, calcium alginate, magnesium alginate, ammonium alginate, esters of alginate, etc.), carboxymethyl cellulose, poly lactic acid, poly glutamic acid, pectin, xanthan, carrageenan, furcellaran, gum arabic, karaya gum, gum ghatti, gum carob and gum tragacanth (see U.S. Provisional Application No. 60/717,200 filed on Sep. 15, 2005, the entire teachings of which are incorporated herein by reference). One or more sweeteners and/or flavorants can be optionally included in the powder formulation.

Though the above description is directed toward routes of oral administration of pharmaceutical compositions consistent with embodiments of the invention, it is understood by those skilled in the art that other modes of administration using vehicles or carriers conventionally employed and which are inert with respect to the pharmaceutically acceptable ferrous iron compounds may be utilized for preparing and administering the pharmaceutical compositions. Illustrative of such methods, vehicles and carriers are those described, for example, in Remington's Pharmaceutical Sciences, 18^{th} ed. (1990), the disclosure of which is incorporated herein by reference.

A “subject” is preferably a human, but can also be another animal in need of treatment with a pharmaceutically acceptable ferrous iron compound as described above, e.g., companion animals (e.g., dogs, cats, and the like), farm animals (e.g., cows, pigs, horses and the like) and laboratory animals (e.g., rats, mice, guinea pigs and the like). As used herein, a subject in need of treatment with a pharmaceutically acceptable ferrous iron compound that includes a ferrous ion include subjects with diseases and/or conditions that can be treated with a pharmaceutically acceptable ferrous iron compound that includes a ferrous ion to achieve a beneficial therapeutic and/or prophylactic result. A beneficial outcome includes a decrease in the severity of symptoms or delay in the onset of symptoms, increased longevity and/or more rapid or more complete resolution of the disease or condition. For example, a subject in need of treatment typically has elevated serum phosphate levels, hyperphosphatemia, resulting from, for example, impaired kidney function or hypoparathyroidism. A subject in need of treatment also includes a subject with chronic renal failure. Other examples of subjects in need of treatment with a pharmaceutically acceptable ferrous iron compound include patients with a disease associated with disorders of phosphate metabolism. Examples of diseases and/or disorders of this type include hyperparathyroidism, inadequate renal function, and hyperphosphatemia.

“Treating,” as used herein includes both therapeutic and prophylactic treatments.

An “effective amount” of a pharmaceutically acceptable ferrous iron compound that includes a ferrous ion or a mixture of pharmaceutically, acceptable ferrous iron compounds is a quantity that results in a beneficial clinical outcome of the condition being treated with the ferrous iron compounds) compared with the absence of treatment. An “effective amount” of a pharmaceutically acceptable ferrous iron compound that includes a ferrous ion or a mixture of such pharmaceutically acceptable ferrous iron compounds is also an amount which achieves a beneficial propylactic effect when given to a subject at risk of development of for example, renal failure, hypoparathyroidism, or hyperphosphatemia, to prevent onset of these diseases and/or conditions. The amount of a pharmaceutically acceptable ferrous iron compound that includes a ferrous ion or a mixture of such pharmaceutically acceptable ferrous iron compounds administered to the subject will depend on the degree, severity, and type of the disease or condition, the amount of therapy desired, and the release characteristics of the pharmaceutical formulation. It will also depend on the subject's health, size, weight, age, sex and tolerance to drugs. Typically, the pharmaceutical compositions of the invention are administered for a sufficient period of time to achieve the desired therapeutic effect. Typically between 0.1 mg per day and 20 g per day of the pharmaceutically acceptable ferrous iron compound or a mixture of the pharmaceutically acceptable ferrous iron compounds (alternatively between 1 mg per day and 10 g per day, between 1 mg per day and 5 g per day, alternatively between 0.5 g per day and 5 g per day, alternatively between 0.5 per day and 3 g per day) is administered to the subject in need of treatment. These dosages can be administered several times per day (e.g., 2, 3, 4 or 5 times per day) or once per day.

An effective amount of a phosphate sequestrant as described above, such as an amine polymer (e.g., sevelamer, colesevelam and colestipol), or a pharmaceutically acceptable lanthanum (e.g., Fosrenol®) or calcium (PhosLo®) salt, is generally known in the art. Also, an effective amount of a phosphate transport inhibitor, an HMG-CoA reductase inhibitor, an alkaline phosphatase inhibitor or a bile acid sequestrant is generally known in the art. When one or more of these second agents, e.g., a phosphate sequestrant, a phosphate transport inhibitor, an HMG-CoA reductase inhibitor, an alkaline phosphatase inhibitor or a bile acid sequestrant, are used in combination with the pharmaceutically acceptable ferrous iron compound, the effective amount of the pharmaceutically acceptable ferrous iron compound is adjusted to take into account the effective amount of the second agent(s) to achieve the desired phosphate binding capacity.

For example, when a pharmaceutical composition of the invention comprises a pharmaceutically acceptable ferrous iron compound as described above and an amine polymer (preferably an aliphatic amine polymer), typically between 5 mg per day and 15 g per day of the pharmaceutical composition (alternatively between 50 mg per day and 12 g per day, alternatively between 0.5 g per day and 12 g per day, alternatively between 1 g per day and 12 g per day, alternatively between 0.5 g per day and 10 g per day, alternatively between 1 g per day and 10 g per day, alternatively between 2 g per day and 10 g, alternatively between 3 g per day and 10 g per day, alternatively between 1 g per day and 8 g per day, alternatively between 2 g per day and 8 g per day, alternatively between 2 g per day and 6 g per day, alternatively between 2 g per day and 5 g per day) is administered to the subject in need of treatment. Frequency of administration is as described above when the pharmaceutically acceptable ferrous iron compound is administered. In one specific example, 0.8-7.2 g (e.g., 1.2 g, 1.6 g, 1.8 g, 2.0, 2.4 g, 3.0 g, 3.2 g, 3.6 g, 4.0 g or 4.8 g per dose for 2-3 times per day, or 3.0 g, 3.2 g, 3.6 g, 4.0 g or 4.8 g, 5.4 g, 6.0 g, 6.2 g, 6.6 g, 7.0 g or 7.2 g per dose for once per day) of the pharmaceutical composition is administered per day. The pharmaceutical compositions can be administered at least four times per day with meals, at least three times per day with meals, at least twice per day with meals, at least once per day with meals, (see U.S. application Ser. No. 11/262,502, filed Oct. 27, 2005, the entire contents of which are incorporated herein by reference).

Typically, the composition of the invention can be administered before or after a meal, or with a meal. Preferably, the effective amount of the composition of the invention is administered several times per day or once per day with a meal. As used herein, “before” or “after” a meal is typically within two hours, preferably within one hour, more preferably within thirty minutes, most preferably within ten minutes of commencing or finishing a meal, respectively. For once per day dosing, the effective amount of the composition of the invention is preferably administered before or after, or with the largest meal of the day.

The pharmaceutically acceptable ferrous iron compound(s) can be administered as multiple dosage units or preferably as a single dosage unit. As used herein a dosage unit may be a tablet, sachet, slurry, food formulation, troche, capsule, elixir, suspension, syrup, wafer, chewing gum or the like prepared by art recognized procedures. Preferably a dosage unit is a tablet, capsule, sachet, slurry, suspension or food formulation, more preferably the dosage unit is a tablet, slurry, suspension or food formulation, most preferably the dosage unit is a tablet or sachet. Typically, the desired dose of a pharmaceutically acceptable ferrous iron compound(s) is administered as multiple tablets or capsules, or a single dose of a sachet, slurry, food formulation, suspension or syrup.

Those skilled in the art will be aware that the amounts of the various components of the pharmaceutical compositions of the invention to be administered in accordance with the method of the invention to a subject will depend upon those factors noted above.

The invention is illustrated by the following examples which are not intended to be limiting in any way.

### EXEMPLIFICATION

Iron compounds that were used in this example, for example, (+)-iron(II) L-ascorbate, iron(II) acetate, iron(II) oxide, iron(II) oxalate, iron (II/III) oxide nanopowder and ferrous carbonate saccharated were obtained from commercial vendors, e.g., Aldrich® Advancing Science, and used without further purification. The iron compounds having a ferrous ion were handled under an inert atmosphere.

### Example 1

### Preparation of a Composition Having Iron(II) Formate

Iron(II) sulfate (275.23 g) was dissolved in 10% formic acid solution (120 mL) with stirring. In a separate flask sodium formate (54 g) was dissolved in 10% formic acid solution (50 mL) with stirring. With stirring, the sodium formate solution was added to the iron(II) sulfate solution. A light blue precipitate was formed. The resulting mixture was stirred at room temperature for 1 hour and filtered. A portion was lyophilized to afford 8.91 g (#1) and another portion was dried at 70° C. in a vacuum oven under nitrogen to afford 34.99 g (#2).

### Example 2

### Preparation of a Mixture of Poly(Acrylic Acid, Sodium Salt) and Iron(II) Salt

To a solution of poly(acrylic acid, sodium salt) (44.44 g of a 45 wt. % aqueous solution) in deionized water (500 mL) was added drop wise a solution of iron(II) chloride (42 g) in deionized water (500 mL). The resulting mixture was filtered, washed with water (100 mL), and dried in a forced-air oven at 60° C. to afford 7.96 g.

### Example 3

### Preparation of a Mixture of Poly(Acrylic Acid, Sodium Salt) and Iron(II) Salt

To a solution of poly(acrylic acid, sodium salt) (50 g) in deionized water (170 mL) was added drop wise a solution of iron(II) chloride tetrahydrate (42.35 g) dissolved in water (75 mL). After stirring over two nights the mixture was dried in a forced-air oven at 60° C. to afford 52.54 g.

### Example 4

### Preparation of a Mixture of Poly(Allylamine) and Iron(II) Salt

To a solution of polyallylamine hydrochloride (PAA.HCl, 50% (w/w) aqueous solution) was added deionized water (1050 g) followed by NaOH (185.38 g of 50% (w/w) NaOH in water) to form a partially neutralized polyallylamine solution. To a portion of the partially neutralized poly(allylamine) solution (110.6 g) was added iron(II) oxide (3.77 g). After stirring at room temperature epichlorohydrin (0.985 mL) was added. A gel was formed after 30 minutes. After curing at room temperature the gel was broken into small pieces and suspended into deionized water (4 L). After stirring for 20 minutes the suspension was filtered. The filtered polymer was washed once more with deionized water (4 L each wash). The filtered polymer was dried in a forced-air oven at 60° C. to afford 20.07 g.

### Example 5

### Preparation of a Mixture of Poly(Allylamine) and Iron(II) Salt

To a partially neutralized poly(allylamine) solution (110.6 g, see Example 4) was added iron(II) oxide (7.54 g). After stirring at room temperature epichlorohydrin (0.985 mL) was added. A gel was formed after 30 minutes. After curing at room temperature the gel was broken into small pieces and suspended into deionized water (4 L). After stirring for 20 minutes the suspension was filtered. The filtered polymer was washed once more with deionized water (4 L each wash). The filtered polymer was dried in a forced-air oven at 60° C. to afford 24.44 g.

### Example 6

### Preparation of a Mixture of Poly(Allylamine) and Iron (II, III) Salt

To a partially neutralized poly(allylamine) solution (110.6 g, see Example 4) was added iron(II, III) oxide (12.16 g). After stirring at room temperature epichlorohydrin (0.985 mL) was added. A gel was formed after 30 minutes. After curing at room temperature the gel was broken into small pieces and suspended into deionized water (4 L). After stirring for 20 minutes the suspension was filtered. The filtered polymer was washed once more with deionized water (4 L each wash). The filtered polymer was dried in a forced-air oven at 60° C. to afford 28.31 g.

### Example 7

### Preparation of a Mixture of Poly(Allylamine) and Iron(II) Salt

To a partially neutralized poly(allylamine) solution (110.6 g, see Example 4) was added iron(II, III) oxide (24.31 g). After stirring at room temperature epichlorohydrin (0.985 mL) was added. A gel was formed after 30 minutes. After curing at room temperature the gel was broken into small pieces and suspended into deionized water (4 L). After stirring for 20 minutes the suspension was filtered. The filtered polymer was washed once more with deionized water (4 L each wash). The filtered polymer was dried in a forced-air oven at 60° C. to afford 40.79 g.

### Example 8

### Preparation of a mixture of hydroxylamine modified poly(2-hydroxyethylacrylate-co-divinylbenzene) and iron(II) salt

A solution of 2-hydroxyethyl acrylate (50 mL), divinylbenzene (1.9 g), and azoisobutyronitrile (0.526 g) in ethanol (136 mL) was heated to 60° C. under a nitrogen atmosphere for 1 hour. The reaction exothermed to 70° C. After cooling to room temperature, the reaction mixture was diluted with diethyl ether, and filtered, to afford 46.2 g poly(2-hydroxyethylacrylate-co-divinylbenzene) after drying.

A suspension of 568-3 poly(2-hydroxyethylacrylate-co-divinylbenzene), deionized water (100 mL), and hydroxylamine (50 mL of 50% aqueous soln) was heated at 55° C. for 24 hours under a nitrogen atmosphere. The reaction mixture was diluted with methanol and filtered, to afford 6.3 g hydroxylamine modified poly(2-hydroxyethylacrylate-co-divinylbenzene) after drying.

Dispersed 6.3 g of ground polymer hydroxylamine modified poly(2-hydroxyethylacrylate-co-divinylbenzene), into 500 ml of stirring deionized water (pH 6.6). Dissolved 29.83 g of Fe(II)Cl₂-4H₂O into 60 ml of deionized water. Slowly pipetted iron solution into the stirring polymer solution. Polymer solution immediately turned a red/orange color, and the pH of the solution was 3.8. The pH was then adjusted upward w/50% NaOH and equilibrated to pH 6.35 after 90 minutes. Solution darkened upon base addition. The dark brown/orange solids were filtered off and placed overnight into the 60° C. forced air oven to dry. Yield 10.93 g of dark brown solid.

### Example 9

### Preparation of a Mixture of Hydroxylamine-Ethyl Acrylate-Modified, Epichlorohydrin-Crosslinked Polyallylamine and Iron(II) Salt

A suspension of epichlorohydrin crosslinked poly(allylamine) (6.05 g) and ethyl acrylate (11.5 mL) in ethanol (200 mL) was stirred at room temperature for 3 days. The mixture was filtered, washed with methanol by suspending the collected solid in methanol, stirring for 1 hour, and filtering. The methanol wash was repeated twice more. Drying in a 60° C. forced afforded 10.57 g of ethyl acrylate modified epichlorohydrin cross linked polyallylamine.

A suspension of ethyl acetate modified epichlorohydrin crosslinked polyallylamine, deionized water (200 mL), and hydroxylamine (25 mL of 50% aqueous soln) was heated at 65° C. for 2 days under a nitrogen atmosphere. The reaction mixture was diluted with methanol and filtered, to afford 7.9 g hydroxylamine-ethyl acrylate-modified epichlorohydrin crosslinked polyallylamine after drying.

Dispersed 7.88 g of ground polymer, hydroxylamine-ethyl acrylate-modified, epichlorohydrin-crosslinked polyallylamine, into 450 ml of stirring deionized water. Dissolved 22.19 g of Fe(II)Cl₂ into 50 ml of deionized water. Slowly pipetted iron solution into the stirring polymer solution (pH 6.0). The pH was adjusted upward w/50% NaOH and equilibrated to 6.5 after 2 hours. The dark brown solids were filtered off and re-suspended into 1 L of deionized water for 30 minutes. This process was repeated one more time. The dark brown solids were filtered off and placed overnight into the 60° C. forced air oven to dry. Yield 10.93 g of brick red solid.

### Example 10

### Preparation of a Mixture of Ethyl Acrylate-Modified Epichlorohydrin Crosslinked Polyallylamine and Iron(II) Salt

A suspension of epichlorohydrin-crosslinked polyallylamine) (6.53 g) and ethyl acrylate (69 mL) in ethanol (200 mL) was stirred at room temperature for 4 days. The mixture was filtered, washed with methanol by suspending the collected solid in methanol, stirring for 1 hour, and filtering. The methanol wash was repeated twice more. Drying in a 60° C. forced afforded 15.9 g of ethyl acrylate modified epichlorohydrin crosslinked polyallylamine.

A suspension of ethyl acrylate modified epichlorohydrin crosslinked polyallylamine (15.2 g), NaOH (48 g of a 50% aqueous soln), deionized water (100 mL), and ethanol (150 mL) was refluxed overnight (temperature=80° C.). The polymer was suspended in deionized water, stirred, and filtered. This process was repeated until the suspension had conductivity 0.88 mS/cm and pH 11.48. The filtered solids were dried in an air-forced oven at 60° C. to afford 13.47 g ethyl acrylate-modified epichlorohydrin crosslinked polyallylamine.

Dispersed 10.0 g of the ground form of the prepared polymer into about 450 mL of stirring deionized water (pH 11.0). 33.0 g of Fe(II)Cl₂-7H₂O was dissolved into 50 ml of deionized water. The iron solution was slowly added into the stirring polymer solution (pH 6.9). The pH of the mixture after 4 hours of stirring was 5.9. The dark brown solids were filtered off and resuspended into 500 mL of deionized water for 30 minutes. This process was repeated one more time. The final pH was 6.5. The dark brown solids were filtered off and placed overnight into the 60° C. forced air oven to dry. Yield 10.39 g of brick red solid.

### Example 11

### Preparation of a Mixture of Modified Epichlorohydrin Crosslinked Polyallylamine and Iron(II) Salt

A solution of poly(allylamine) (10.0 g), N-(3-dimethylaminopropyl)-N′-ethylcarbodimide hydrochloride (7.34 g), 1-hydroxybezotriazole (4.47 g), and 3,4-dihydroxybenzoic acid (6.01 g) in water (100 mL) was stirred at room temperature for 24 hours. Dialysis of the reaction solution, and drying at 60° C. in a forced air oven afforded 20 g.

10.0 g of the ground form of the prepared polymer was dissolved into 40 ml of stirring deionized water. The mixture was capped and placed into the 60° C. forced air oven for several minutes to fully dissolve solid materials therein (pH about 10.2), and then taken out and cooled to room temperature. 1.2 mL of epichlorohydrin was added. A gel was formed within 30 minutes. The gel was allowed to be cured overnight. Block gel was broken up into smaller pieces and suspended into about 2 L of deionized water for 30 minutes. The mixture was filtered, and then the filtered solid was resuspended into about 2 L of methanol for 30 minutes. The mixture was then again filtered and resuspended into 2 L of de-ionized water for 30 minutes (pH about 8.8). The pH of the suspension was adjusted upwards with 50% NaOH and was allowed to reach an equilibrium pH of about 9.5. A solution of 20.0 g FeCl₂-4H₂O in 100 mL of de-ionized water was slowly pipetted into the above swollen gel. After 15 minutes, the pH of the mixture was 4.9. The mixture was then filtered. The filtered solid was then resuspended into about 2 L of deionized water for 30 minutes. This procedure was repeated one more time. The final pH of the resuspended solution was about 6.8. The dark brown solids were filtered off and placed overnight into the 60° C. forced air oven to dry. Yield 11.0 g of brick red solid.

### Example 12

### Preparation of a Mixture of Epichlorohydrin Crosslinked Poly(Allylamine) and Iron(II) Salt (1:1 Wt/Wt)

To a stirred suspension of 9.8 mol % epichlorohydrin crosslinked poly(allylamine) (6.00 g) in deionized water (80 g) was added iron(II) acetate (6.00 g dissolved in 30 mL deionized water). The resulting suspension was lyophilized to afford 11.83 g.

### Example 13

### Preparation of a Mixture of Epichlorohydrin Crosslinked Poly(Allylamine) and Iron(II) Salt: (1:1.5 Wt/Wt)

To a stirred suspension of 9.8 mol % epichlorohydrin crosslinked poly(allylamine) (6.00 g) in deionized water (80 g) was added iron(II) acetate (9.00 g). The resulting suspension was lyophilized to afford 14.19 g.

### Example 14

### Preparation of a Mixture of Epichlorohydrin Crosslinked Poly(Allylamine) and Iron(II) Salt: (2:1 Wt/Wt)

To a stirred suspension of 9.8 mol % epichlorohydrin crosslinked poly(allylamine) (6.00 g) in deionized water (80 g) was added iron(II) acetate (3.00 g). The resulting suspension was lyophilized to afford 9.35 g.

### Example 15

### Effects of Compounds for Reducing Urinary Phosphate Levels

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats was distributed into groups.

Purina 5002 was used as the standard diet. The compound or compound mixture being tested was mixed with Purina 5002 to result in a final concentration 0.5% (or as indicated in the table) by weight. Cellulose at 0.5% by weight was used as a negative control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated using Excel program. A summary of comparison of the amounts of urinary phosphate obtained from the test rats is shown in Table 1.

TABLE 1

| | | Urinary | |
|---|---|---|---|
| Tested | % of | Phosphate | % |
| compound(s) | Diet | (mg/day) | Control |
| Example 15 | 0.7 | 13.3 | 65.4 |
| Example 8 | 0.5 | 18.0 | 88.6 |
| Example 9 | 0.5 | 17.0 | 65.6 |
| Example 2 | 0.5 | 20.1 | 77.7 |
| Example 3 | 0.5 | 15.9 | 91.4 |
| Example 10 | 0.5 | 23.1 | 109.3 |
| (+)-Iron(II) L- | 0.5 | 13.4 | 78.6 |
| ascorbate* | | | |
| Iron(II) acetate* | 0.50 | 12.8 | 65.9 |
| Iron(II)oxide* | 0.50 | 20.2 | 104.3 |
| Iron(II) oxalate* | 0.50 | 14.8 | 89.0 |
| Iron(II/III) oxide | 0.50 | 16.7 | 86.7 |
| nanopowder* | | | |
| Ferrous Carbonate, | 1.00 | 13.1 | 88.1 |
| saccharated* | | | |
| Example 11 | 0.50% | 14.0 | 75.3 |
| Iron(II) Acetate* | 0.70% | 8.0 | 44.4 |
| Iron(II) Acetate* | 0.50% | 12.4 | 69.1 |
| Iron(II) Acetate* | 0.30% | 12.1 | 67.4 |
| Fe(II) | 0.50% | 10.4 | 55.6 |
| acetylacetonato* | | | |
| Example 12 | 0.50 | 7.9 | 45.9 |
| Example 13 | 0.50 | 15.3 | 80.1 |
| Example 14 | 0.50 | 13.4 | 70.4 |

*from Aldrich ® Advancing Science.

As shown in Table 1, the amounts of urinary phosphate obtained from the rats which went through the iron therapy were much lower than that of a control which did not go through the iron therapy.

What is claimed is:

1. A pharmaceutical composition, comprising:
a) a pharmaceutically acceptable ferrous iron compound selected from the group consisting of iron(II) acetate, iron(II) citrate, iron(II) ascorbate, iron(II) oxalate, iron(II) oxide, iron(II) carbonate, iron(II) carbonate saccharated, iron(II) acetylacetonate, iron(II) formate, iron(II) sulfate, iron(II) chloride and combinations thereof; and
b) a pharmaceutically acceptable carrier.

2. The pharmaceutical composition of claim 1, wherein the composition is essentially free of ferric ion.

3. The pharmaceutical composition of claim 1, wherein the ferrous ion of the ferrous iron compound comprises 5-35% by anhydrous weight of the pharmaceutical composition.

4. The pharmaceutical composition of claim 1, wherein the ferrous iron compound is selected from the group consisting of iron(II) oxide, iron(II) acetate, iron(II) citrate, iron(II) ascorbate, iron(II) oxalate and combinations thereof.

5. A pharmaceutical composition, comprising:
a) a pharmaceutically acceptable ferrous iron compound;
b) an amine polymer or a pharmaceutically acceptable salt thereof; and
c) a pharmaceutically acceptable carrier.

6. The pharmaceutical composition of claim 5, wherein the ferrous iron compound is selected from the group consisting of iron(II) acetate, iron(II) citrate, iron(II) ascorbate, iron(II) oxalate, iron(II) oxide, iron(II) carbonate, iron(II) carbonate saccharated, iron(II) acetylacetonate, iron(II) formate, iron(II) sulfate, iron(II) chloride and combinations thereof.

7. The pharmaceutical composition of claim 5, wherein the composition is essentially free of ferric ion.

8. The pharmaceutical composition of claim 5, wherein the amine polymer is an aliphatic amine polymer.

9. The pharmaceutical composition of claim 8, wherein the aliphatic amine polymer comprises one or more repeat units represented by a structural formula selected from: 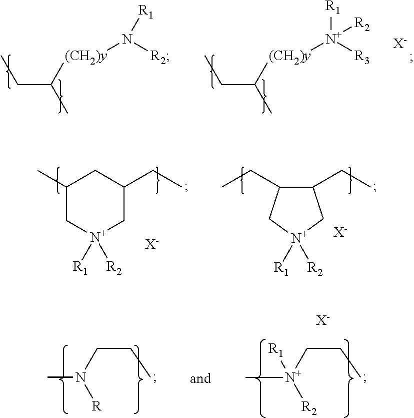
or a salt thereof, wherein:
y is independently zero or an integer from one to ten;
R, R₁, R₂ and R₃, independently, are H, a substituted or unsubstituted alkyl group or an aryl group; and
X⁻ is an exchangeable negatively charged counterion.

10. The pharmaceutical composition of claim 9, wherein the aliphatic amine polymer is crosslinked.

11. The pharmaceutical composition of claim 10, wherein the ferrous iron compound is entrained within the crosslinked aliphatic amine polymer.

12. The pharmaceutical composition of claim 10, wherein the aliphatic amine polymer is crosslinked with a bifunctional crosslinking agent.

13. The pharmaceutical composition of claim 12, wherein the crosslinking agent is present in an amount from about 0.5-35% by weight, based upon total weight of aliphatic amine monomer plus crosslinking agent.

14. The pharmaceutical composition of claim 13, wherein the aliphatic amine polymer is a crosslinked polyallylamine.

15. The pharmaceutical composition of claim 14, wherein the polyallylamine polymer is sevelamer.

16. The pharmaceutical composition of claim 14, wherein the polyallylamine polymer is a chloride salt of sevelamer.

17. The pharmaceutical composition of claim 14, wherein the polyallylamine polymer is a carbonate salt of sevelamer.

18. The pharmaceutical composition of claim 14, wherein the polyallylamine polymer is a mixed chloride and carbonate salt of sevelamer.

19. The pharmaceutical composition of claim 14, wherein the polyallylamine is colesevelam.

20. The pharmaceutical composition of claim 14, wherein the ferrous iron compound is selected from the group consisting of iron(II) oxide, iron(II) acetate, iron(II) citrate, iron(II) ascorbate, iron(II) oxalate and combinations thereof.

21. The pharmaceutical composition of claim 5, wherein the pharmaceutical composition is a tablet.

22. A method of treating hyperphosphatemia in a subject, comprising administering to the subject an effective amount of a pharmaceutically acceptable ferrous iron compound selected from the group consisting of iron(II) acetate, iron(II) citrate, iron(II) ascorbate, iron(II) oxalate, iron(II) oxide, iron(II) carbonate, iron(II) carbonate saccharated, iron(II) acetylacetonate, iron(II) formate, iron(II) sulfate, iron(II) chloride and combinations thereof.

23. The method of claim 22, wherein the ferrous iron compound is essentially free of ferric ion.

24. The method of claim 22, wherein the pharmaceutically acceptable ferrous iron compound is administered in an amount of between 0.1 mg/day and 10 g/day.

25. The method of claim 22, wherein a mixture of ferrous iron compounds is administered to the subject.

26. The method of claim 22, wherein the ferrous iron compound is selected from the group consisting of iron(II) oxide, iron(II) acetate, iron(II) citrate, iron(II) ascorbate, iron(II) oxalate and combinations thereof.

27. The method of claim 22, wherein the method further comprises co-administering a phosphate sequestrant to the subject.

28. The method of claim 27, wherein the phosphate sequestrant is selected from the group consisting of a pharmaceutically acceptable calcium compound, a pharmaceutically acceptable aluminum compound, a pharmaceutically acceptable magnesium compound, a pharmaceutically acceptable lanthanum compound and a pharmaceutically acceptable zinc compound.

29. The method of claim 22, wherein the method further comprises co-administering to the subject a drug selected from the group consisting of a phosphate transport inhibitor, an HMG-CoA reductase inhibitor, an alkaline phosphatase inhibitor and a bile acid sequestrant.

30. A method of treating hyperphosphatemia in a subject, comprising administering to the subject an effective amount of a pharmaceutical composition comprising:
a) a pharmaceutically acceptable ferrous iron compound; and
b) an aliphatic amine polymer or a pharmaceutically acceptable salt thereof.

31. The method of claim 30, wherein the ferrous iron compound is selected from the group consisting of iron(II) acetate, iron(II) citrate, iron(II) ascorbate, iron(II) oxalate, iron(II) oxide, iron(II) carbonate, iron(II) carbonate saccharated, iron(II) acetylacetonate, iron(II) formate, iron(II) sulfate, iron(II) chloride and combinations thereof.

32. The method of claim 30, wherein the ferrous iron compound is essentially free of ferric ion.

33. The method of claim 31, wherein a mixture of ferrous iron compounds is administered to the subject.

34. The method of claim 30, wherein the amine polymer is an aliphatic amine polymer.

35. The method of claim 33, wherein the aliphatic amine polymer comprises one or more repeat units represented by a formula selected from the group consisting of: 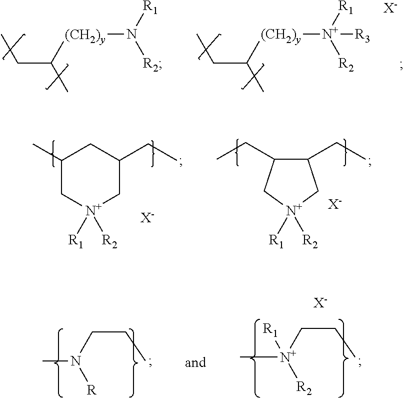
or a salt or a copolymer thereof, wherein:
y is zero or an integer from one to ten;
R, R₁, R₂ and R₃, independently, is H, a substituted or unsubstituted alkyl group or an aryl group; and
X⁻ is an exchangeable negatively charged counterion.

36. The method of claim 35, wherein the aliphatic amine polymer is a polyallylamine crosslinked by means of a multifunctional cross-linking agent.

37. The method of claim 36, wherein the ferrous iron compound is entrained within the crosslinked polyallylamine.

38. The method of claim 36, wherein the polyallylamine is sevelamer.

39. The method of claim 36, wherein the polyallylamine is colesevelam.

40. The method of claim 36, wherein the ferrous iron compound is selected from the group consisting of iron(II) oxide, iron(II) acetate, iron(II) citrate, iron(II) ascorbate, iron(II) oxalate and combinations thereof.

41. The method of claim 30, wherein the pharmaceutically acceptable ferrous iron compound is administered in an amount of between 0.1 mg/day and 10 g/day.

42. The method of claim 30, wherein the ferrous ion of the ferrous iron compound comprises 5-35% by anhydrous weight of the pharmaceutical composition.

43. The method of claim 30, wherein the method further comprises co-administering a phosphate sequestrant to the subject, the phosphate sequestrant being selected from the group consisting of a pharmaceutically acceptable calcium compound, a pharmaceutically acceptable aluminum compound, a pharmaceutically acceptable magnesium compound, a pharmaceutically acceptable lanthanum compound and a pharmaceutically acceptable zinc compound.

44. The method of claim 30, wherein the method further comprises co-administering to the subject a drug selected from the group consisting of a phosphate transport inhibitor, an HMG-CoA reductase inhibitor, an alkaline phosphatase inhibitor and a bile acid sequestrant.

45. The method of claim 30, wherein the method further comprises co-administering to the subject a drug selected from the group consisting of a phosphate transport inhibitor, an HMG-CoA reductase inhibitor, an alkaline phosphatase inhibitor and a bile acid sequestrant.
